(19) United States
(12) Patent Application Publication
(10) Pub. No.: US 2025/0143975 A1
(43) Pub. Date: May 8, 2025

(54) NASOGASTRIC TUBING FIXATION DEVICE

(71) Applicant: Leigh Reed, Tifton, GA (US)

(72) Inventor: Leigh Reed, Tifton, GA (US)

(21) Appl. No.: 18/387,924

(22) Filed: Nov. 8, 2023

(51) Int. Cl. A61J 15/00; A61M 25/02

(52) U.S. Cl. CPC A61J 15/0061; A61J 15/0003; A61M 2025/0226

(57) ABSTRACT

A nasogastric tubing fixation device for retaining a position of a nasogastric tube relative to a nasal cavity of a nose includes a band with a front side, a back side, and a peripheral edge that is attached to and extends between the front side and the back side. The band is flexible so the band can complement a curvature of the nose when the band is positioned on the nose. An adhesive coating covers the back side to releasably retain the band on the nose. A pair of straps coupled to the band engages the nasogastric tube. Each of the pair of straps comprises a first end attached to the peripheral edge, a second end distal to the first end, and a middle portion extending between the first end and the second end. The middle portion is flexible whereby the middle portion can bend upwardly toward the band.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical tubing holders and more particularly pertains to a new medical tubing holder for retaining a position of a nasogastric tube relative to a nasal cavity.

### (2) Description of Related Art Including Information Disclosed Under 37 CFR 1.97 and 1.98

The prior art relates to medical tubing holders. More specifically, the prior art relates to devices and methods for retaining a position of a nasogastric tube that is inserted into a nasal cavity of a patient. Nasogastric tubes may be inserted through the nasal cavity into the stomach of a patient, for example to decompress the stomach or administer nutrition or medication to patients who are unable to tolerate oral intake. Safely securing the tube at a particular position with respect to the patient is important for the tube to function properly and for patient comfort. Typically, medical staff secure nasogastric tubes with tape. However, tape does not hold the tube in place very well, so the tube may become dislodged, removed, or repositioned after insertion. Thus, there is a need in the art for a device that can stabilize a positioning of a nasogastric tube within a nasal cavity of a patient. Such a device should be comfortable for the patient to wear and convenient for the medical staff to use.

### BRIEF SUMMARY OF THE INVENTION

An embodiment of the disclosure meets the needs presented above by generally comprising a nasogastric tube that is configured to be inserted into a nasal cavity of a nose of a patient. A band has a front side, a back side, and a peripheral edge that is attached to and extends between the front side and the back side. The band comprises a flexible material whereby the band is configured to complement a curvature of the nose when the band is positioned on the nose. An adhesive coating covers the back side of the band to releasably retain the band on the nose. A pair of straps is coupled to the band for engaging the nasogastric tube. Each of the pair of straps comprises a first end that is attached to the peripheral edge, a second end that is distal to the first end, and a middle portion that is attached to and extends between the first end and the second end. The middle portion is flexible whereby the middle portion is positionable to bend upwardly toward the band to engage the nasogastric tube thereby stabilizing a position of the nasogastric tube within the nasal cavity.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric view of a nasogastric tubing fixation device according to an embodiment of the disclosure.

FIG. 2 is a rear isometric view of an embodiment of the disclosure.

FIG. 3 is a front view of an embodiment of the disclosure.

FIG. 4 is a side view of an embodiment of the disclosure.

FIG. 5 is an in-use view of an embodiment of the disclosure.

### DETAILED DESCRIPTION OF THE INVENTION

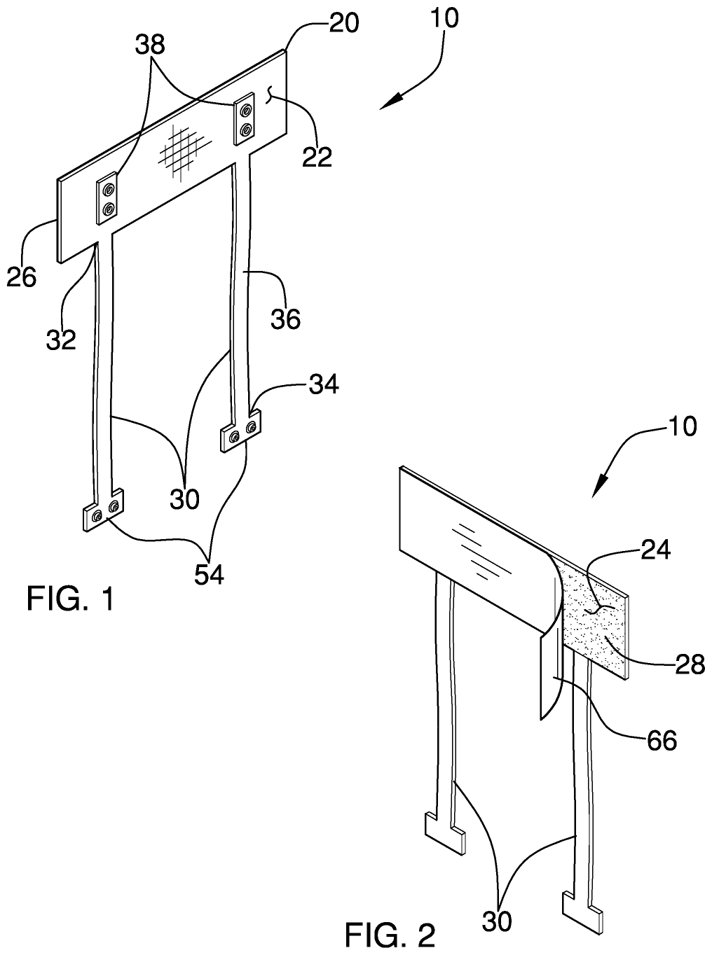

With reference now to the drawings, and in particular to FIGS. 1 through 5 thereof, a new medical tubing holder embodying the principles and concepts of an embodiment of the disclosure and generally designated by the reference numeral 10 will be described.

As best illustrated in FIGS. 1 through 5, the nasogastric tubing fixation device 10 is generally compatible with a nasogastric tube 12 that is configured to be inserted into a nasal cavity 14 of a nose 16 of a patient 18. The nasogastric tubing fixation device 10 generally comprises a band 20 having a front side 22, a back side 24, and a peripheral edge 26 that is attached to and extends between the front side 22 and the back side 24. The band 20 generally comprises a flexible material whereby the band 20 is configured to complement a curvature of the nose 16 when the band 20 is positioned on the nose 16. As shown in FIG. 1, the band 20 may have a rectangular shape.

An adhesive coating 28 covers the back side 24 of the band 20. The adhesive coating 28 is generally configured to releasably retain the band 20 on the nose 16. As shown in FIG. 2, a cover 66 may be removably coupled to the adhesive coating 28, for example to protect the adhesive coating 28 from damage or degradation before the band 20 is positioned on the nose 16.

A pair of straps 30 is coupled to the band 20 for engaging the nasogastric tube 12. The pair of straps 30 is configured to stabilize a position of the nasogastric tube 12 within the nasal cavity 14. In embodiments, each of the pair of straps 30 generally comprises a first end 32 that is attached to and extends downwardly from the peripheral edge 26. A second end 34 is distal to the first end 32. A middle portion 36 is attached to and extends between the first end 32 and the second end 34.

The middle portion 36 is flexible whereby the middle portion 36 is positionable to bend and extend upwardly toward the band 20 to engage the nasogastric tube 12. The second end 34 is thereby positionable adjacent to the band 20 such that the second end 34 is couplable to the band 20, for example when the band 20 is positioned on the nose 16. The middle portion 36 can thus engage the nasogastric tube 12 so that the pair of straps 30 is configured to stabilize the position of the nasogastric tube 12 within the nasal cavity 14.

A pair of band fasteners 38 may be coupled to the front side 22 of the band 20. In such embodiments, the nasogastric tube fixation device 10 may further include a pair of strap fasteners 54. Each of the pair of strap fasteners 54 is couplable to a respective one of the pair of straps 30. Each of the pair of strap fasteners 54 is complementary to an associated one of the pair of band fasteners 38 such that each of the pair of strap fasteners 54 releasably couples the respective one of the pair of straps 30 to the band 20.

The pair of band fasteners 38 may comprise a first panel 40 that is positioned above a first strap of the pair of straps 42. A first panel mating member 44 may be attached to the first panel 40. A second panel 46 may be positioned above a second strap of the pair of straps 48. A second panel mating member 50 is attached to the second panel 46.

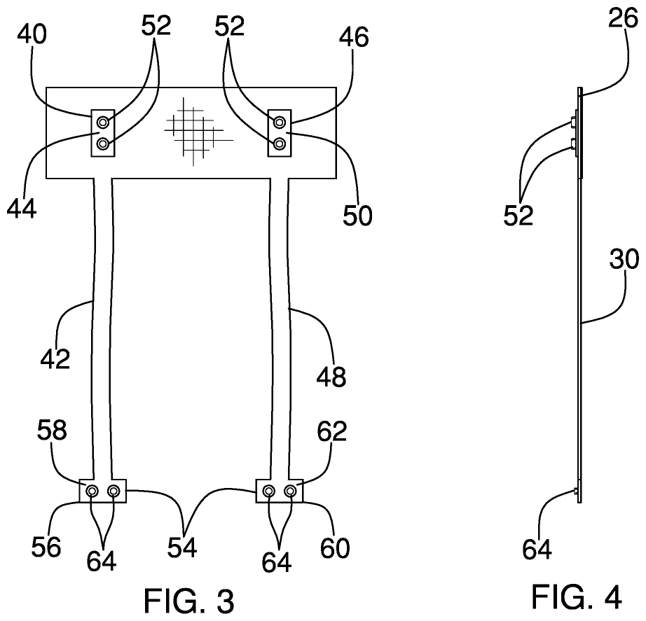

For example, each of the first panel mating member 44 and the second panel mating member 50 may comprise a pair of receivers 52. Each receiver of the pair of receivers 52 may be aligned with the other along a longitudinal plane. For example, as shown in FIG. 3, the pair of receivers 52 on the first panel 40 are stacked vertically on top of each other and are aligned with the first strap 42, which extends downwardly from the band 20 beneath the pair of receivers 52. Similarly, the pair of receivers 52 on the second panel 46 are stacked vertically on top of each other and are aligned with the second strap 48, which extends downwardly from the band 20 beneath the pair of receivers 52. By engaging the pair of receivers 52, the first strap 42 may be releasably couplable to the first panel 40 and the second strap 48 may be releasably couplable to the second panel 46.

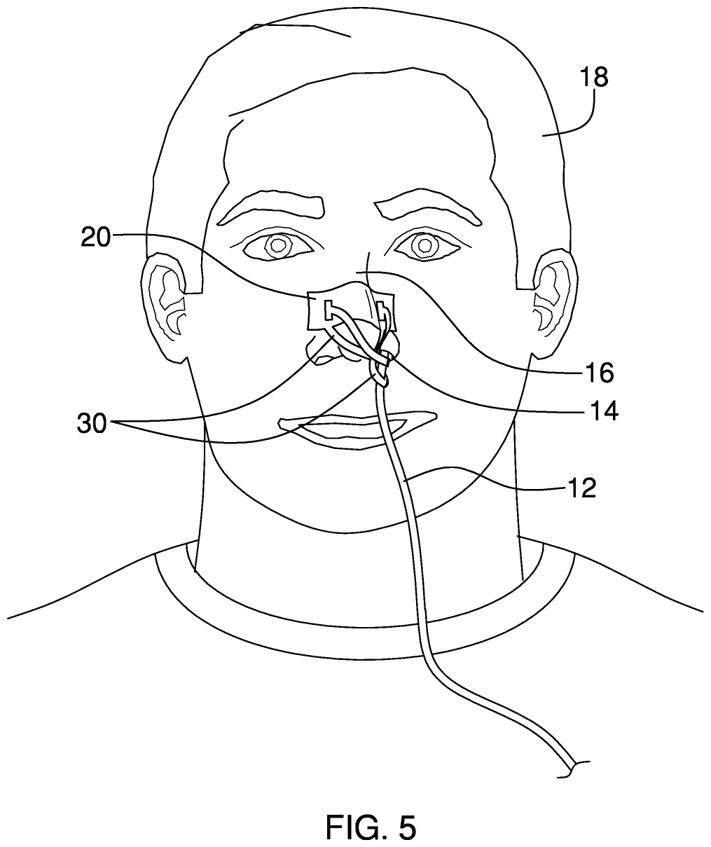

In some embodiments, the first strap 42 crosses over the second strap 48 when the middle portion 36 of each of the first strap 42 and the second strap 48 bends upwardly toward the band 20 to engage the nasogastric tube 12. An example of this embodiment is provided in FIG. 5.

The pair of strap fasteners 54 may generally comprise a first flange 56 that is attached to the second end 34 of the first strap 42 and a second flange 60 that is attached to the second end 34 of the second strap 48. A first flange mating member 58 may be attached to the first flange 56 and a second flange mating member 62 may be attached to the second flange 60. Each of the first flange 56 and the second flange 60 may be rectangular such that each of the pair of straps 30 forms a T-shape.

For example, each of the first flange mating member 58 and the second flange mating member 62 comprising a pair of gibs 64. The pair of gibs 64 are generally complementary to the pair of receivers 52 such that the pair of gibs 64 engages the pair of receivers 52 to couple each of the pair of strap fasteners 54 to the associated one of the pair of band fasteners 38 thereby releasably coupling the respective one of pair of straps 30 to the band 20.

Each gib of the pair of gibs 64 may be aligned with the other along a transverse plane. For example, as shown in FIG. 3, the pair of gibs 64 on the first flange 56 are positioned next to each other and are aligned along a transverse, or horizontal, plane. In other words, the pair of gibs 64 on the first flange 56 are positioned at a 90.0° rotation with respect to the pair of receivers 52 on the first panel 40. Similarly, the pair of gibs 64 on the second flange 60 are positioned next to each other and are aligned along a transverse, or horizontal, plane such that the pair of gibs 64 on the second flange 60 are positioned at a 90.0° rotation with respect to the pair of receivers 52 on the second panel 46.

In some embodiments, the first panel 40 has a rectangular shape that is complementary to a rectangular shape of the first flange 56 and the second panel 46 has a rectangular shape that is complementary to a rectangular shape of the second flange 60. The first panel 40 may be positioned at a 90.0° rotation with respect to the first flange 56 and the second panel 46 may be positioned at a 90.0° rotation with respect to the second flange 60.

In some examples, each of the pair of band fasteners 38 and each of the pair of strap fasteners 54 comprise snaps.

In use, a user may insert the nasogastric tube 12 into the nasal cavity 14 of the patient 18. The user may adhere the back side 24 of the band 20 to the nose 16 of the patient 18 and wrap the pair of straps 30 around the nasogastric tube 12 to secure, stabilize, or fix the position of the nasogastric tube 12 with respect to the nasal cavity 14. The nasogastric tubing fixation device 10 can thus inhibit the nasogastric tube 12 from being unintentionally removed from or repositioned with respect to the patient 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical tubing fixation system comprising:
a nasogastric tube being configured to be inserted into a nasal cavity of a nose of a patient;
a band having a front side, a back side, and a peripheral edge being attached to and extending between the front side and the back side, the band comprising a flexible material whereby the band is configured to complement a curvature of the nose when the band is positioned on the nose; and
an adhesive coating covering the back side of the band, the adhesive coating being configured to releasably retain the band on the nose;
a pair of straps being coupled to the band for engaging the nasogastric tube, the pair of straps being configured to stabilize a position of the nasogastric tube within the nasal cavity, each of the pair of straps comprising:
a first end being attached to the peripheral edge;
a second end being distal to the first end; and
a middle portion being attached to and extending between the first end and the second end, the middle portion being flexible whereby the middle portion is positionable to bend upwardly toward the band to engage the nasogastric tube.

2. The medical tubing fixation system of claim 1, wherein the second end is positionable adjacent to the band such that the second end is couplable to the band when the band is positioned on the nose whereby the pair of straps is configured to stabilize the position of the nasogastric tube within the nasal cavity.

3. The medical tubing fixation system of claim 1, wherein the band has a rectangular shape.

4. The medical tubing fixation system of claim 1, further comprising:
a pair of band fasteners being coupled to the front side of the band; and
a pair of strap fasteners, each of the pair of strap fasteners being coupled to a respective one of the pair of straps, each of the pair of strap fasteners being complementary to an associated one of the pair of band fasteners such that each of the pair of strap fasteners releasably couples the respective one of the pair of straps to the band.

5. The medical tubing fixation system of claim 4, wherein each of the pair of band fasteners and each of the pair of strap fasteners comprises snaps.

6. The medical tubing fixation system of claim 4, the pair of band fasteners further comprising:
a first panel being positioned above a first strap of the pair of straps;
a first panel mating member being attached to the first panel;
a second panel being positioned above a second strap of the pair of straps;
a second panel mating member being attached to the second panel; and
each of the first panel mating member and the second panel mating member comprising a pair of receivers, each of the pair of receivers engaging an associated one of the pair of strap fasteners.

7. The medical tubing fixation system of claim 6, wherein the first strap of the pair of straps is releasably couplable to the first panel and the second strap of the pair of straps is releasably couplable to the second panel.

8. The medical tubing fixation system of claim 6, wherein the first strap of the pair of straps crosses over the second strap of the pair of straps when the middle portion of each of the first strap of the pair of straps and the second strap of the pair of straps bends upwardly toward the band to engage the nasogastric tube.

9. The medical tubing fixation system of claim 6, wherein each of the pair of receivers is aligned with the other along a longitudinal plane.

10. The medical tubing fixation system of claim 6, the pair of strap fasteners further comprising:
a first flange being attached to the second end of the first strap of the pair of straps;
a first flange mating member being attached to the first flange;
a second flange being attached to the second end of the second strap of the pair of straps;
a second flange mating member being attached to the second flange; and
each of the first flange mating member and the second flange mating member comprising a pair of gibs, the pair of gibs being complementary to the pair of receivers such that the pair of gibs engages the pair of receivers to couple each of the pair of strap fasteners to the associated one of the pair of band fasteners thereby releasably coupling the respective one of pair of straps to the band.

11. The medical tubing fixation system of claim 10, wherein each of the pair of gibs being aligned with the other along a transverse plane.

12. The medical tubing fixation system of claim 10, wherein the first panel has a shape being complementary to the first flange and the second panel has a shape being complementary to a shape of the second flange.

13. The medical tubing fixation system of claim 12, wherein each of the first panel and the second panel is positioned at a 90.0° rotation with respect to each of the first flange and the second flange.

14. The medical tubing fixation system of claim 1, further comprising a cover being removably coupled to the adhesive coating.

15. A medical tubing fixation system comprising:
a nasogastric tube being configured to be inserted into a nasal cavity of a nose of a patient;
a band having a front side, a back side, and a peripheral edge being attached to and extending between the front side and the back side, the band comprising a flexible material whereby the band is configured to complement a curvature of the nose when the band is positioned on the nose, the band being a rectangular shape;
an adhesive coating covering the back side of the band, the adhesive coating being configured to releasably retain the band on the nose;
a pair of straps being coupled to the band for engaging the nasogastric tube, the pair of straps being configured to stabilize a position of the nasogastric tube within the nasal cavity, each of the pair of straps comprising:
a first end being attached to and extending downwardly from the peripheral edge;
a second end being distal to the first end;
a middle portion being attached to and extending between the first end and the second end, the middle portion being flexible whereby the middle portion is positionable to bend and extend upwardly toward the band to engage the nasogastric tube, and wherein the second end is positionable adjacent to the band such that the second end is couplable to the band when the band is positioned on the nose whereby the pair of straps is configured to stabilize the position of the nasogastric tube within the nasal cavity;
a pair of band fasteners being coupled to the front side of the band, the pair of band fasteners comprising:
a first panel being positioned above a first strap of the pair of straps;
a first panel mating member being attached to the first panel;
a second panel being positioned above a second strap of the pair of straps;
a second panel mating member being attached to the second panel; and
each of the first panel mating member and the second panel mating member comprising a pair of receivers, each receiver of the pair of receivers being aligned with the other along a longitudinal plane;
wherein the first strap of the pair of straps is releasably couplable to the first panel and the second strap of the pair of straps is releasably couplable to the second panel;
wherein the first strap of the pair of straps crosses over the second strap of the pair of straps when the middle portion of each of the first strap of the pair of straps and the second strap of the pair of straps bends upwardly toward the band to engage the nasogastric tube;
a pair of strap fasteners, each of the pair of strap fasteners being coupled to a respective one of the pair of straps, each of the pair of strap fasteners being complementary to an associated one of the pair of band fasteners such that each of the pair of strap fasteners releasably couples the respective one of the pair of straps to the band, the pair of strap fasteners comprising:
a first flange being attached to the second end of the first strap of the pair of straps;
a first flange mating member being attached to the first flange;
a second flange being attached to the second end of the second strap of the pair of straps;
a second flange mating member being attached to the second flange;
each of the first flange mating member and the second flange mating member comprising a pair of gibs, each gib of the pair of gibs being aligned with the other along a transverse plane, the pair of gibs being complementary to the pair of receivers such that the pair of gibs engages the pair of receivers to couple each of the pair of strap fasteners to the associated one of the pair of band fasteners thereby releasably coupling the respective one of pair of straps to the band;
each of the first flange and the second flange being rectangular such that each of the pair of straps forms a T-shape;
wherein the first panel has a rectangular shape being complementary to a rectangular shape of the first flange and the second panel has a rectangular shape being complementary to a rectangular shape of the second flange;
wherein the first panel is positioned at a 90.0° rotation with respect to the first flange and the second panel is positioned at a 90.0° rotation with respect to the second flange;
wherein each of the pair of band fasteners and each of the pair of strap fasteners comprises a snap; and
a cover being removably coupled to the adhesive coating.
